(19) United States
(12) Patent Application Publication
(10) Pub. No.: US 2008/0294200 A1
(43) Pub. Date: Nov. 27, 2008

(54) Spinous process implants and methods of using the same

(76) Inventors: Andrew Kohm, Burlingame, CA (US); Tom Slater, Mountain View, CA (US); Hugues F. Malandain, Mountain View, CA (US); Arthur S. Hsieh, Sunnyvale, CA (US)

Correspondence Address: COATS & BENNETT/MEDTRONIC, 1400 CRESCENT GREEN, SUITE 300, CARY, NC, 27518, US

(21) Appl. No.: 11/802,931

(22) Filed: May 25, 2007

(51) Int. Cl. A61B 17/70; A61B 17/58; A61F 2/44

(52) U.S. Cl. 606/279; 623/17.11; 606/248

(57) ABSTRACT

Devices and methods for performing a procedure within a spine are disclosed herein. In one embodiment, a method includes disposing an implant adjacent a side of a spinous process. A first portion of the implant is placed over a top side of the spinous process in a lateral direction. A second portion of the implant is placed under a bottom side of the spinous process in a lateral direction such that at least a portion of the spinous process is disposed within an interior region defined by the implant. In another embodiment, an apparatus includes an implant configured to be coupled to a spinous process. The implant has an outer surface configured to contact at least one of a second implant or an interspinous-process spacer. A closure member is coupled to the implant and has an open configuration and a closed configuration to secure the implant to the spinous process.

### CROSS-REFERENCE TO RELATED APPLICATIONS

This application is related to U.S. patent application, entitled “Spinous Process Implants And Methods Of Using The Same,” Attorney Docket No. KYPH-036/01US 305363-2187, filed on same date, the entire disclosure of which is hereby incorporated by reference in its entirety.

### BACKGROUND

The invention relates generally to medical devices and procedures, including, for example, medical devices and methods for percutaneous treatment of spinal conditions, and more particularly, to the treatment of spinal compression using percutaneous spinal implants that can be coupled to adjacent spinous processes.

A back condition that impacts many individuals is spinal stenosis. Spinal stenosis is a progressive narrowing of the spinal canal that causes compression of the spinal cord. Each vertebra in the spinal column has an opening that extends through it and is aligned vertically with other vertebra openings to form the spinal canal. The spinal cord runs through the spinal canal. As the spinal canal narrows, the spinal cord and nerve roots extending from the spinal cord and between adjacent vertebrae are compressed and may become inflamed. Spinal stenosis can cause pain, weakness, numbness, burning sensations, tingling, and in particularly severe cases, may cause loss of bladder or bowel function, or paralysis. The legs, calves and buttocks are most commonly affected by spinal stenosis, however, the shoulders and arms may also be affected.

Mild cases of spinal stenosis may be treated with rest or restricted activity, non-steroidal anti-inflammatory drugs (e.g., aspirin), corticosteroid injections (epidural steroids), and/or physical therapy. Some patients find that bending forward, sitting or lying down may help relieve the pain. This may be due to bending forward creates more vertebral space, which may temporarily relieve nerve compression. Because spinal stenosis is a progressive disease, the source of pressure may have to be surgically corrected (decompressive laminectomy) as the patient has increasing pain. The surgical procedure can remove bone and other tissues that have impinged upon the spinal canal or put pressure on the spinal cord. Two adjacent vertebrae may also be fused during the surgical procedure to correct an area of instability, improper alignment or slippage, such as that caused by spondylolisthesis. Surgical decompression can relieve pressure on the spinal cord or spinal nerve by widening the spinal canal to create more space. This procedure requires that the patient be given a general anesthesia and an incision is made in the patient to access the spine to remove the areas that are contributing to the pressure. This procedure, however, may result in blood loss and an increased chance of significant complications, and usually results in an extended hospital stay.

Some known procedures involve the implantation of a device (e.g., an interspinous process implant) between the spinous processes to limit the extension between the adjacent spinous processes. Such devices are typically in direct contact with the spinous processes at least during some of the movements of the spinal column of the patient.

A need exists for an apparatus that can be used in the treatment of spinal conditions, and that can reduce or eliminate potential damage to a spinous process as a result of a device implanted between adjacent a spinous process to limit extension.

### SUMMARY OF THE INVENTION

Devices and methods for performing a procedure within a spine are disclosed herein. In one embodiment, a method includes disposing an implant adjacent a side of a spinous process. A first portion of the implant is placed over a top side of the spinous process in a lateral direction. A second portion of the implant is placed under a bottom side of the spinous process in a lateral direction such that at least a portion of the spinous process is disposed within an interior region defined by the implant. In another embodiment, an apparatus includes an implant configured to be coupled to a spinous process. The implant has an outer surface configured to contact at least one of a second implant or an interspinous-process spacer. A closure member is coupled to the implant and has an open configuration to place the implant on the spinous process and a closed configuration to secure the implant to the spinous process.

### BRIEF DESCRIPTION OF THE DRAWINGS

FIG. 1 is a schematic illustration of a medical device according to an embodiment of the invention shown coupled to a schematic representation of a portion of a spine.

FIG. 2 is a side perspective view of an implant according to an embodiment of the invention.

FIG. 3 is an end view of the implant of FIG. 2.

FIG. 4 is a rear view of adjacent vertebrae and a pair of implants according to an embodiment of the invention each shown coupled to one of the adjacent vertebrae, and a device disposed between the implants.

FIG. 5 is a side view of the implants, device and vertebrae of FIG. 4.

FIG. 6 is an exploded end view of an implant according to an embodiment of the invention.

FIG. 7 is a side view of adjacent vertebrae and a pair of implants according to an embodiment of the invention each shown coupled to one of the adjacent vertebrae and contacting each other.

FIG. 8 is a rear view of the implants and adjacent vertebrae of FIG. 7 shown with the implants at a spaced distance from each other.

FIG. 9 is a side view of the implants and adjacent vertebrae of FIG. 8 shown with the implants at a spaced distance from each other.

FIG. 10 is an end view of an implant according to an embodiment of the invention shown coupled to a portion of a spinous process.

FIG. 11 is a side perspective view of a portion of the implant of claim 10.

FIGS. 12-14 are each an end view of a different embodiment of an implant.

FIG. 15 is a rear view of three implants according to an embodiment of the invention shown coupled to three adjacent vertebrae.

FIG. 16 is side view of the implants and adjacent vertebra of FIG. 15.

FIG. 17 is a rear view of two implants according to an embodiment of the invention shown coupled to two adjacent vertebrae.

FIG. 18 is side view of the implants and adjacent vertebra of FIG. 17.

FIG. 19 is a side perspective view of an implant of FIG. 18.

FIG. 20 is a side view of an implant according to an embodiment of the invention shown in a collapsed configuration and disposed within a portion of an insertion sheath.

FIG. 21 is a rear view of the implant of FIG. 20 shown in an expanded configuration and coupled to a vertebra.

FIG. 22 is a rear view of another embodiment of an implant shown in an expanded configuration and coupled to a vertebra.

FIG. 23 is a side perspective view of adjacent vertebrae and a pair of implants according to an embodiment of the invention each shown coupled to one of the adjacent vertebrae.

FIG. 24 is a rear view of an implant according to another embodiment of the invention.

FIG. 25 is a side view of a portion of a spinal column illustrating three vertebrae and an opening in a spinal ligament (interspinous ligament) both above and below the middle vertebra.

FIG. 26 is a rear view of the portion of the spinal column of FIG. 25 and the implant of FIG. 24.

FIG. 27 is a rear view of the portion of the spinal column of FIG. 26 (the spinal ligament not shown) and the implant of FIG. 24 disposed on the spinous process of the middle vertebra.

FIG. 28 is a rear view of a pair of vertebrae and a pair of implants according to an embodiment of the invention each shown coupled to one of the adjacent vertebrae.

FIG. 29 is a rear view of a pair of vertebrae and a pair of implants according to an embodiment of the invention each shown coupled to one of the adjacent vertebrae.

FIG. 30 is a rear view of a pair of spinous processes each with an implant according to an embodiment of the invention shown coupled to the spinous processes.

FIGS. 31-33 are each a rear view of a spinous process with an implant according to different embodiments of the invention shown coupled to the spinous processes.

FIG. 34 is a rear view of a pair of spinous processes each with an implant according to an embodiment of the invention shown coupled to the spinous processes

FIG. 35 is a rear view of a spinous process with an implant according to an embodiment of the invention shown coupled to the spinous process.

FIG. 36 is a side view of the spinous process and implant of FIG. 35.

FIG. 37 is a rear view of an implant according to an embodiment of the invention shown with a linking member in an open position.

FIG. 38 is a rear view of the implant of FIG. 37 shown with a linking member in a closed position.

FIG. 39 is a rear view of the implant of FIG. 37 shown in a deformed configuration.

FIG. 40 is a rear view of an implant according to an embodiment of the invention.

FIG. 41 is a rear view of an implant according to an embodiment of the invention.

FIG. 42 is a flowchart illustrating a method according to an embodiment of the invention.

FIG. 43 is a flowchart illustrating a method according to another embodiment of the invention.

FIG. 44 is a flowchart illustrating a method according to another embodiment of the invention.

### DETAILED DESCRIPTION

Devices and methods for performing medical procedures within a spine are disclosed herein. In one embodiment, an apparatus includes a two-part implant having a first implant and a second implant. Each of the implants can be coupled to an adjacent spinous process. In some embodiments, the implants have a surface that can contact a device disposed between the adjacent spinous processes, such as an extension limiting interspinous process implant. In some embodiments, the first implant has a surface that can be in and out of contact with a surface of the second implant when each of the implants is coupled to respective adjacent spinous processes. In some embodiments, the first and second implants can contact each other when the spinal column is in extension to limit the amount of extension and be at a spaced distance from each other during flexion of the spinal column. Thus, when the implants are coupled to adjacent spinous processes, flexion of the spinal column can still occur, as the implants do not fixedly couple the vertebrae and spinous processes to each other.

In some embodiments, the implants can at least partially limit extension of the spinal column and/or at least partially limit flexion of the spinal column. For example, in some embodiments a first implant can be coupled to a first spinous process and a second implant can be coupled to a second spinous process adjacent the first spinous process, and a linking member, such as a tether, rope or lever, can be coupled to both the first and second implants. The linking member can be used to limit the amount flexion between the two spinous processes.

In some embodiments, an implant is substantially U-shaped and can be coupled to a bottom or top side of a spinous process. In some embodiments, an implant is substantially C-shaped and can be coupled to a side portion of a spinous process. In some embodiments, an implant covers a rear-most portion of a spinous process.

The implants can provide protection from wear or damage to a spinous process that can result, for example, from contact by a device disposed between adjacent spinous processes. For example, an interspinous process implant can be disposed between two adjacent spinous processes to limit the extension of the spinal column. Such devices may rub against and/or impact the spinous processes during movement of the spinal column. The implants can function as a protective cap in conjunction with such a device. In some embodiments, the implants can collectively function to limit the extension of the spinal column as an interspinous process implant and also as a protective cover for the spinous processes. In some embodiments, the implant includes only a single implant that can be coupled to a spinous process. In such an embodiment, the implant can include a surface that can contact a device disposed between the spinous process and an adjacent spinous process.

In one embodiment, an apparatus includes an implant configured to be coupled to a first spinous process of a spinal column. The implant has a substantially C-shape when coupled to the first spinous process and includes an outer surface configured to contact a device disposed between the first spinous process and a second spinous process of the spinal column when the spinal column is in extension. The outer surface is configured to be at a spaced distance from the device when the spinal column is in flexion.

In another embodiment, an apparatus includes a first implant configured to be coupled to a first spinous process of a spinal column. A second implant is configured to be coupled to a second spinous process of the spinal column and a linking member is coupled to the first implant and the second implant. A surface of the first implant is configured to contact at least one of the second implant or an interspinous-process implant when the spinal column is in extension and be at a spaced distance from the at least one of the second implant or the interspinous process implant when the spinal column is in flexion. The linking member is configured to at least partially limit a space between the first spinous process and the second spinous process when the spinal column is in flexion.

In another embodiment, an apparatus includes an implant configured to be coupled to a spinous process of a spinal column and has an outer surface configured to contact at least one of a second implant or an interspinous-process spacer. A closure member is coupled to the implant. The closure member has an open configuration to place the implant on the spinous process and a closed configuration to secure the implant to the spinous process.

It is noted that, as used in this written description and the appended claims, the singular forms “a,” “an” and “the” include plural referents unless the context clearly dictates otherwise. Thus, for example, the term “a lumen” is intended to mean a single lumen or a combination of lumens. Furthermore, the words “proximal” and “distal” refer to direction closer to and away from, respectively, an operator (e.g., surgeon, physician, nurse, technician, etc.) who would insert the medical device into the patient, with the tip-end (i.e., distal end) of the device inserted inside a patient's body. Thus, for example, the catheter end inserted inside a patient's body would be the distal end of the catheter, while the catheter end outside a patient's body would be the proximal end of the catheter.

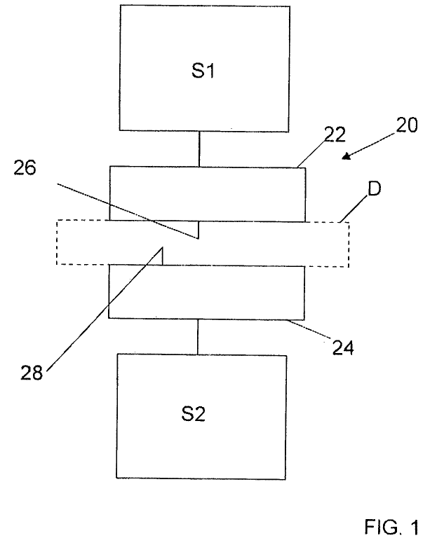

FIG. 1 is a schematic illustration of an example of a medical device that can be used to perform the methods described herein. A medical device 20 can be used, for example, to perform minimally-invasive surgical procedures such as a percutaneous medical procedure within, for example, a spinal column. The medical device 20 includes an implant 22 that can be coupled to a spinous process. In some embodiments, the medical device 20 can include a first implant 22 and a second implant 24 that can each be coupled to a different spinous process. For example, the first implant 22 can be coupled to a first spinous process S1 and the second implant 24 can be coupled to an adjacent second spinous process S2.

The first implant 22 can include an outer surface 26 and the second implant 24 can include an outer surface 28. At least a portion of the outer surface 26 of the first implant 22 and at least a portion of the outer surface 28 of the second implant 24 can each contact a device D that is disposed between the spinous process S1 and the spinous process S2. For example, when the spinal column is in extension, at least a portion of the outer surfaces 26, 28 of the respective implants 22, 24 can contact the device D. When the spinal column is in flexion, the outer surface 26 or the outer surface 28 may not contact the device D. The first implant 22 and the second implant 24 can function as a protective cover to reduce potential damage to the spinous processes caused by contact of the device D with the spinous processes S1 and S2.

In some embodiments, the outer surface 26 and the outer surface 28 are can move in and out of contact with each other. In such embodiments, the device D is not present. For example, the first implant 22 and the second implant 24 can be coupled to the adjacent spinous processes S1 and S2, respectively and be configured such that during normal movement of the spinal column of the patient, the outer surface 26 and the outer surface 28 can come into and out of contact with each other. In such an embodiment, during normal movement of the patient's spine, the outer surface 26 and the outer surface 28 can sometimes be in contact with each other, and at other times be at a spaced distance from each other. For example, during extension of the spinal column, the outer surface 26 and the outer surface 28 can contact each other and limit the extension of the spinous processes S1 and S2 (e.g. movement toward each other). During flexion of the spinal column, the outer surface 26 and the outer surface 28 can be at a spaced distance from each other. Thus, the first implant 22 and the second impact member 24 can limit the extension of the spinous processes S1 and S2, but do not limit the flexion, lateral bending, or axial rotation of the spinous processes S1 and S2 relative to each other or with respect to the spinal column during movement of the spinal column.

The implants 22 and 24 can be coupled to the respective spinous processes S1 and S2, using a variety of different coupling methods. In addition, each of the first implant 22 and the second implant 24 can include the same coupling configurations or have different coupling configurations. In some embodiments, the implant 22 and/or the implant 24 can include a coupling portion on an inner surface that can engage a surface of a spinous process. For example, the inner surface can include spines, protrusions, barbs, etc., that can secure the implant to a spinous process. In some embodiments, the first implant 22 and/or the second implant 24 can define one or more openings through which a screw, nail, pin, or other fastening device can be inserted, and coupled to the spinous process. In other embodiments, the first implant 22 and/or the second implant 24 can be adhesively coupled to a spinous process or coupled by crimping the first implant 22 and/or second implant 24 to the spinous process. In still other embodiments, a component, such as a strap, can be used to couple the first implant 22 and/or the second implant 24 to a spinous process.

The first implant 22 and/or the second implant 24 can be configured to be coupled to only a portion of the spinous process, such as to a lower surface of a superior spinous process, or to an upper surface of an inferior spinous process. In other embodiments, the first implant 22 and/or the second implant 24 can substantially surround a portion of a spinous process. The various configurations of the implants 22, 24 are described in more detail below with reference to specific embodiments.

In some cases, only a single implant 22, 24 is provided and can be inserted into a patient's spinal column and coupled to a selected spinous process. In other cases, two or more implants 22, 24 may be desired. For example, two implants may be desired where a single device (e.g., extension limiting device, interspinous process implant, etc.) is disposed between adjacent spinous processes, with each implant coupled to one of the adjacent spinous processes. In other cases, more than two implants may be desired. For example, a procedure may include the insertion of a first device between a first spinous process and a second spinous process, and a second extension limiting device between, for example, the second spinous process and a third spinous process. In such a case, three implants may be desired, one for each spinous implant that can come into contact with the device. Other quantities of extension limiting devices and implants can alternatively be used, depending on the particular medical condition and the type of treatment desired.

Having described above various general examples, several examples of specific embodiments are now described. These embodiments are only examples, and many other configurations of a medical device 20 are contemplated.

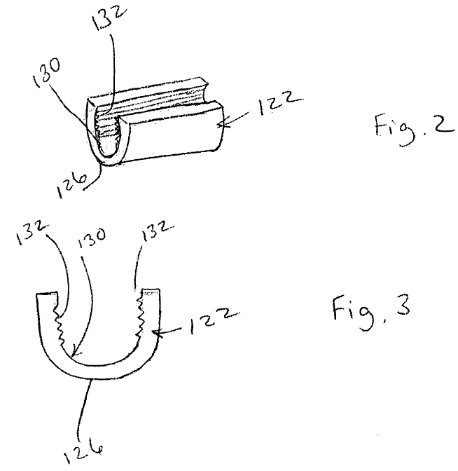

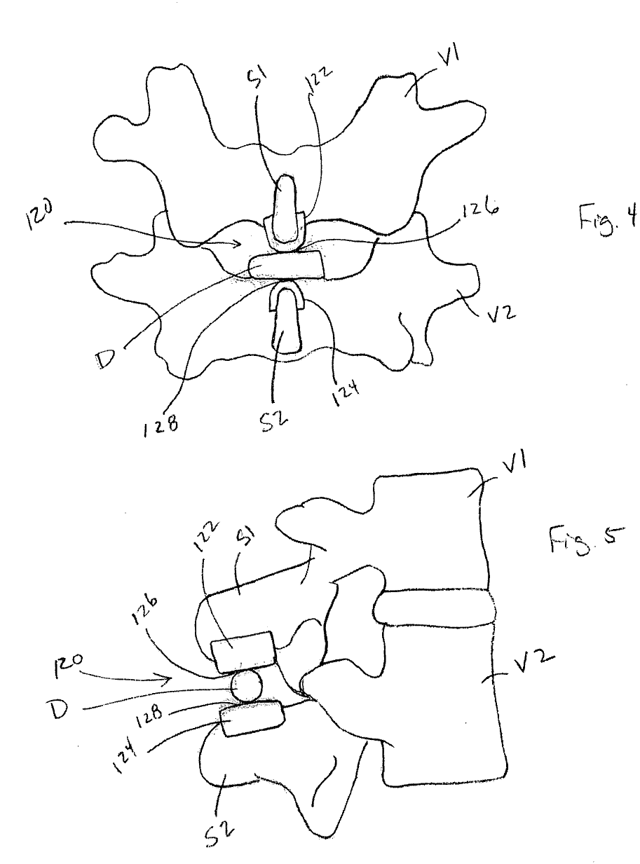

FIGS. 2-5 illustrate a medical device (also referred to as “implant”) according to an embodiment of the invention. A medical device 120 includes a first implant 122 (FIGS. 2-5) and a second implant 124 (FIGS. 4-5). Only the first implant 122 is described with reference to FIGS. 2 and 3; the second implant 124 is identical to the first implant 122, but oriented differently and coupled to a different spinous process. The first implant 122 has a substantially U-shaped configuration and includes an inner surface 130 and an outer surface 126. The inner surface 130 includes a pair of coupling portions 132 configured to couple the first implant 122 to a spinous process. For example, the coupling portions 132 include protrusions that can hook or dig into a spinous process. Alternatively, the inner portion defined by the first implant 122 can be smaller than a size of the spinous process so that the first implant 122 has an interference fit with the spinous process and the coupling portions 132 have a roughened surface to reduce slippage. The outer surface 126 can contact a device disposed between adjacent spinous processes as described in more detail with reference to FIGS. 4 and 5.

As shown in FIGS. 4 and 5, the first implant 122 can be coupled to a first spinous process S1 of a vertebra V1, and the second implant 124 can be coupled to an adjacent second spinous process S2 of a vertebra V2. To secure the first implant 122 to the first spinous process S1, the first implant 122 can be crimped onto the spinous process such that the protrusions of the coupling portions 132 secure the first implant 122 to the first spinous process S1. The second implant 124 can be secured to the second spinous process S2 in the same manner.

As shown in FIGS. 4 and 5, an interspinous-process implant D is disposed between the first implant 122 and the second implant 124. At least a portion of the outer surface 126 of the first implant 122 and at least a portion of an outer surface 128 of the second implant 124 can contact the device D during movement of the spinal column. For example, as stated previously, when the spinal column is in extension, the outer surfaces 126, 128 can contact a surface of the device D as shown in FIGS. 4 and 5. When the spinal column is in flexion, the outer surfaces 126, 128 can be at a spaced distance from the device D (not shown), or can be in contact with the device D. The first implant 122 and the second implant 124 function as a protective cover to help reduce potential damage that can occur as a result of impact of the device D on the first implant 122 and/or second implant 124.

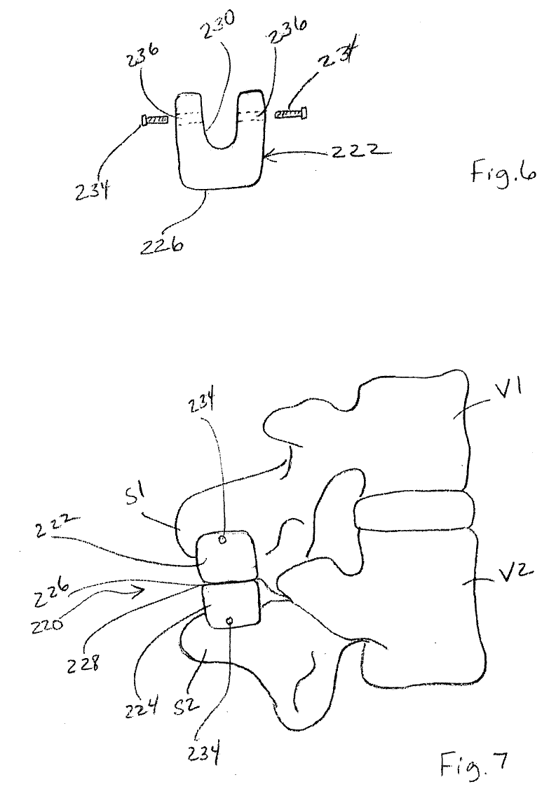

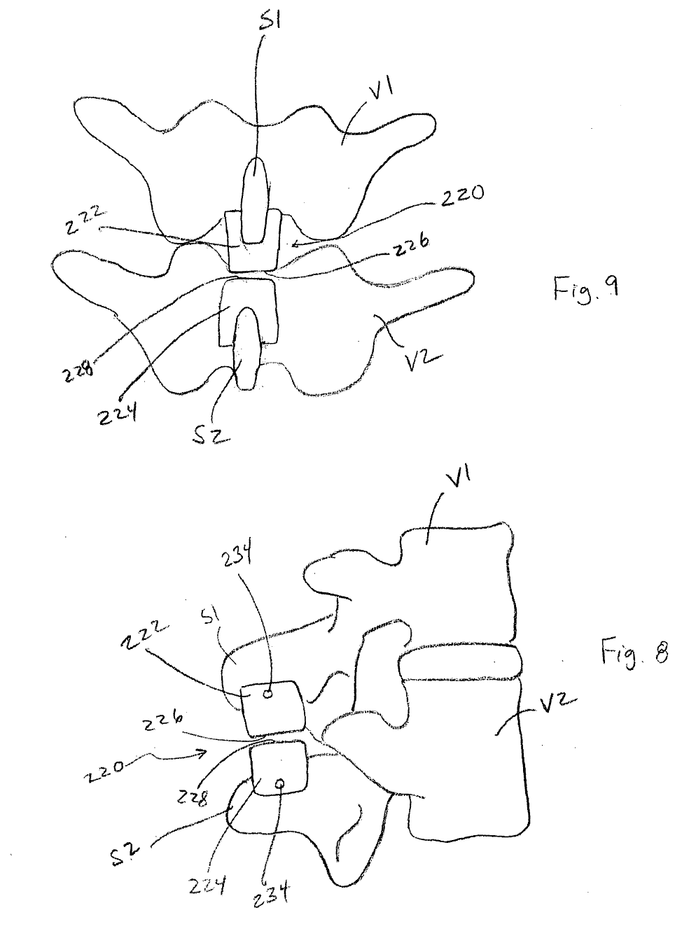

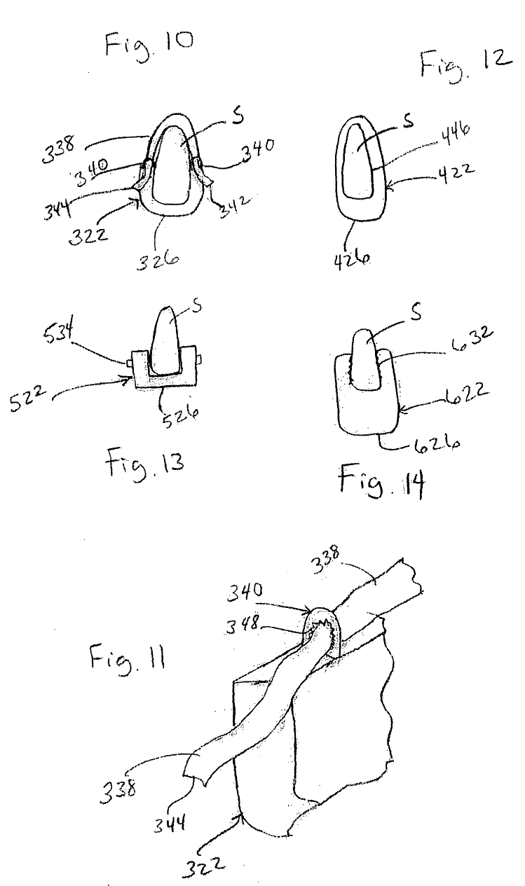

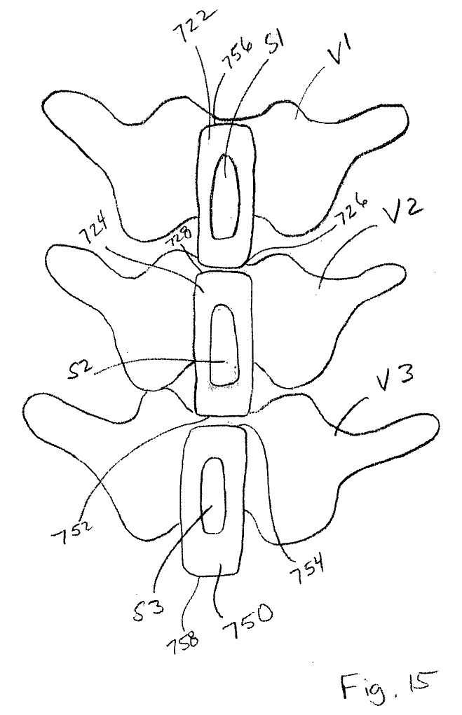

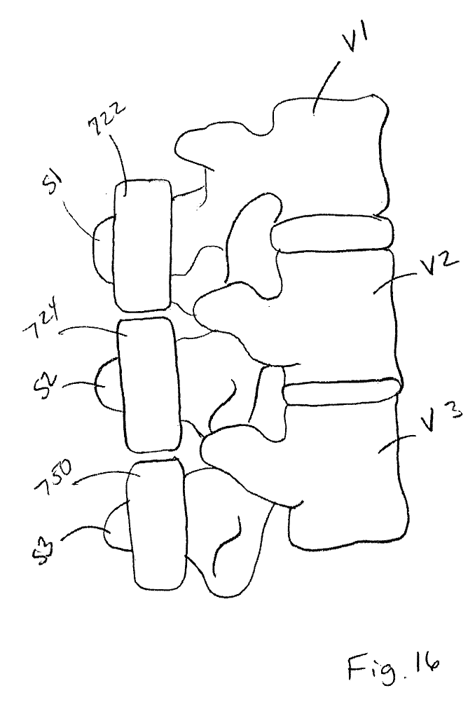

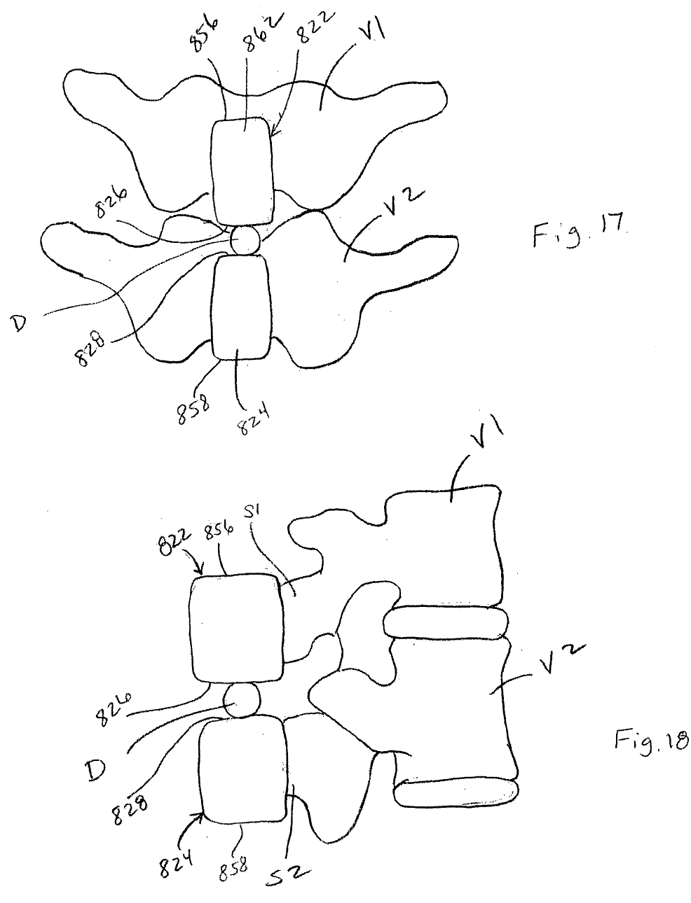

FIGS. 6-9 illustrate a medical device according to another embodiment. A medical device 220 includes a first implant 222 and a second implant 224. Only the first implant 222 is described with reference to FIG. 6; the second implant 224 is identical to the first implant 222, but oriented differently and is coupled to a different spinous process. The first implant 222 includes an inner surface 230 and an outer surface 226. At least a portion of the inner surface 230 is configured to contact a spinous process. At least a portion of the outer surface 226 can contact an outer surface 228 (FIGS. 7-9) of the second implant 224 during movement of a spinal column. In this embodiment, the first implant 222 and the second implant 224 each define openings 236 (shown for first implant 222 in FIG. 6) that can receive therethrough screws 234 that can be used to couple the implant 222 to a spinous process. The openings can be threaded to threadedly engage the screw 234. In other embodiments, the openings are not threaded. In such an embodiment, a self-tapping screw, or a pin or nail can be used to secure the implant to a spinous process.

FIGS. 7-9 illustrate the first implant 222 coupled to a first spinous process S1 of a vertebra V1, and the second implant 224 coupled to a second spinous process S2 of a vertebra V2. To secure the first implant 222 to the first spinous process S1, the first implant is placed over a portion of the first spinous process S1 and the screws 234 are inserted through the openings 236 and threaded at least partially into the first spinous process S1. The second implant 224 is secured to the second spinous process in the same manner. In this embodiment, during extension of the spinal column, the outer surface 226 of the first implant 222 and the outer surface 228 of the second implant 224 can contact each other as shown in FIG. 7. In this manner, the first implant 222 and the second implant 224 function as an extension limiting device (e.g., interspinous-process implant) to limit the amount of extension of the first spinous process S1 and the second spinous process S2. During flexion of the spinal column, the outer surface 226 of the first implant 222 and the outer surface 228 of the second implant 224 can be at a spaced distance from each other as shown in FIGS. 8 and 9. Because the first implant 222 and the second implant 224 are coupled directly to spinous processes S1 and S2, respectively, the potential wear that can occur from an interspinous-process implant impacting the spinous processes S1 and S2 can be reduced.

FIGS. 10-13 illustrate different alternative embodiments of an implant. FIGS. 10 and 11 illustrate an implant 322 having a substantially U-shaped configuration and an outer surface 326. The outer surface 326 can contact a device (e.g., interspinous-process implant) disposed between adjacent spinous processes as described above. Alternatively, the outer surface 326 can contact an outer surface of another implant coupled to an adjacent spinous process. In this embodiment, the implant is couplable to a spinous process S using a strap 338. The strap 338 can be secured to the implant 322 by inserting ends 342 and 344 of the strap 338 through openings 348 (see FIG. 11) of connector 340 coupled to the implant 322. As shown in FIG. 11, one or both of the openings 348 can include teeth or barbs that allow the strap to be pulled through the opening 348 in only one direction. To secure the implant 322 to the spinous process S, the strap 338 can be placed around a portion of the spinous process S and then at least one of the ends, 342 and 344 can be pulled such that the strap 338 tightens the implant 322 to the spinous process S. The implant can include more than two connectors 340 such that additional straps 338 can be used to secure the implant 322 to a spinous process. The connectors 340 can be coupled to the implant 322 using various coupling methods. For example, the connectors 340 can be insert molded into the implant 322 or can be adhesively coupled to the implant 322.

FIG. 12 illustrates an implant 422 having an outer surface 426 that can contact a device disposed between the spinous process S and an adjacent spinous process (not shown). Alternatively, the outer surface 426 can contact another implant coupled to an adjacent spinous process. In this embodiment, the implant 422 defines an opening 446 having a shape substantially the same as a spinous process. The implant 422 can be secured to a spinous process S by placing the opening 446 of the implant 422 over a portion of the spinous process S. The implant 422 can be formed with a flexible material such that the implant 422 can substantially conform to the shape of the spinous process S. In some embodiment, the implant 422 can be sized such that a friction fit or an interference fit is achieved between the implant 422 and the spinous process S. In other embodiments, an adhesive can be used to couple the implant 422 the spinous process S. In some embodiments a threaded coupling or protrusions on an inner surface of the implant 422 as previously described can be used to couple the implant 422 to the spinous process S.

FIG. 13 illustrates an embodiment of an implant 522 that is substantially U-shaped, and has a substantially planar outer surface 526. As with other embodiments, the outer surface 526 can contact a device disposed between adjacent spinous processes or another implant coupled to an adjacent spinous process. In this embodiment, the implant 522 is coupled to a spinous process S using screws 534 as previously described or a single screw passing through the spinous process. It should be understood, however, that other coupling methods can be used.

FIG. 14 illustrates an implant 622 having an outer surface 626 and an inner surface that includes protrusions 632. The outer surface 626 can contact a device disposed between adjacent spinous processes or another implant coupled to an adjacent spinous process. This embodiment is similar to the implant 222, except in this embodiment, the implant can be coupled to a spinous process S using the protrusions 632 as described previously.

FIGS. 15 and 16 illustrate another embodiment of an implant, three of which are shown each coupled to an adjacent spinous process of a spinal column. This embodiment illustrates the use of multiple implants that each surround a portion of the spinous process. A first implant 722 is coupled to a first spinous process S1 of a vertebra V1. A second implant 724 is coupled to a second spinous process S2 of a vertebra V2. A third implant 750 is coupled to a third spinous process S3 of a vertebra V3.

The first implant 722 includes an outer surface 726 that can contact an outer surface 728 of the second implant 724. The second implant 724 also includes an outer surface 752 that can contact an outer surface 754 of the third implant 750. The first implant 722 also includes an outer surface 756 that can contact an outer surface of another implant (optional, not shown) that can be coupled to a spinous process of a superior vertebra (not shown). The third implant 750 includes an outer surface 758 that can contact an outer surface of another implant (optional, not shown) that can be coupled to a spinous process of an inferior vertebra (not shown). As described previously, the outer surfaces of the implants 722, 724, 750 can contact each other when the spinal column is in extension and can be out of contact with each other such that they are at a spaced distance from each other when the spinal column is in flexion.

The first implant 722, second implant 724 and third implant 750 can each be secured to the respective spinous process with a variety of different coupling methods as described herein, such as with adhesives, screws, pins, nails, protrusions, crimping, or a friction fit. The same coupling methods or different coupling methods can be used for each implant. Although the first implant 722, second implant 724, and third implant 750 are each shown having the same configuration, other combinations of implants can be used. For example, in some embodiments, a first implant can be configured to be coupled to only an inferior side of a first spinous process, for example, similar to implants 122 or 222 shown in FIGS. 4 and 9, respectively; a second implant can be configured to surround a second inferior spinous process, for example, similar to the embodiments of FIG. 15 or 17, and a third implant can be configured to be secured to only a superior side of a spinous process inferior to the second spinous process, for example similar to implant 124 or 234.

FIGS. 17-19 illustrate an implant according to another embodiment of the invention. In this embodiment, an implant surrounds a portion of the spinous process and also covers an end of the spinous process. An implant 822 is shown coupled to a first spinous process S1 (FIG. 18) of a vertebra V1 of a spinal column. An implant 824 is shown coupled to a second spinous process S2 (FIG. 18) of a vertebra V2 of the spinal column.

The first implant 822 includes an outer surface 826 that can contact a device D, such as an interspinous-process implant, disposed between the first spinous process S1 and the second spinous process S2. The first implant 822 also includes an outer surface 856 that can contact another device (optional, not shown), for example, that is disposed between the first spinous process S1 and a superior spinous process (not shown). The second implant 824 includes an outer surface 828 that can contact the device D, and an outer surface 858 that can contact a device (optional, not shown), for example, that is disposed between the second spinous process S2 and an inferior spinous process (not shown).

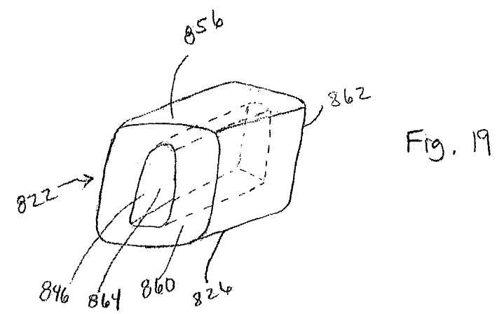

The first implant 822 is described in more detail with reference to FIG. 19; the second implant 824 is identical to the first implant 822, but oriented differently and coupled to a different spinous process. The first implant 822 includes a distal end wall 862 (as best viewed in FIG. 17), and a proximal end wall 860. The proximal end wall 860 defines an opening 846 in fluid communication with an interior space 864 of the first implant 822. The first implant 822 can be coupled to the first spinous process S1 by placing the first implant 822 over the first spinous process S1 such that a portion of the first spinous process S1 is disposed within the interior space 864 defined by the first implant 822.

The first implant 822 can be secured to the first spinous process S1 with a variety of different coupling methods as described herein, such as with adhesives, screws, pins, nails, protrusions, crimping, or a friction fit. As stated above, the second implant 824 is constructed the same as the first implant 822 and can also be coupled to the second spinous process S2 using any of the variety of different coupling methods described herein. As with the previous embodiment, more than two implants can be used in conjunction with, for example, multiple interspinous process implants.

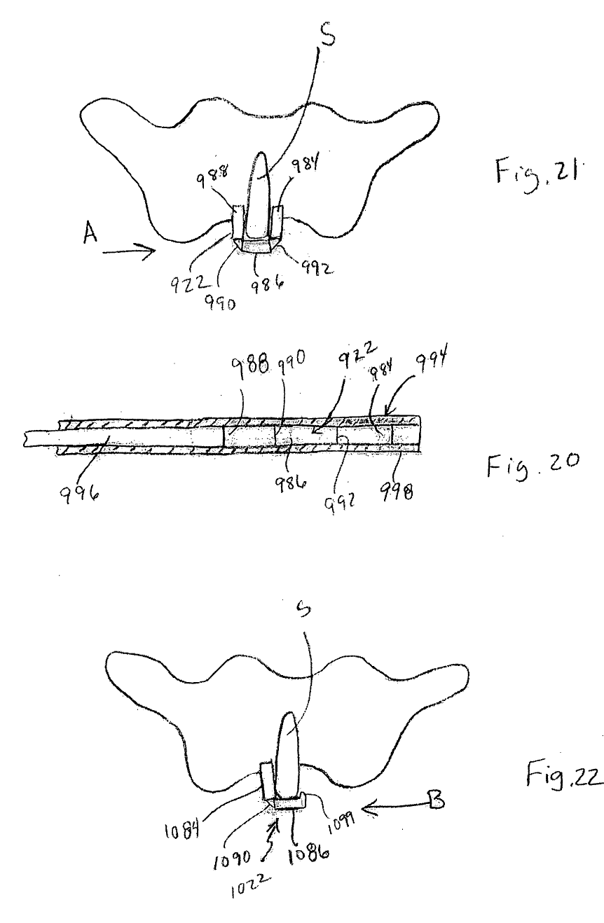

FIGS. 20 and 21 illustrate an embodiment of an implant being movable between a first configuration for insertion of the implant into a spinal column, and a second configuration for coupling the implant to a spinous process. An implant 922 includes a first portion 984 coupled to a second portion 986 at a first joint 992, and a third portion 988 coupled to the second portion 986 at a second joint 990. The first joint 992 and the second joint 990 can each include, for example, a biasing member such as a spring, to bias the implant 922 into the expanded configuration. The implant 922 can be moved to a collapsed configuration by placing a sheath or cannula 994 over the implant 922, which will substantially straighten the implant 922 as shown in FIG. 21. In alternative embodiments, the implant can be formed of a shape-memory material allowing the implant to be formed such that it is biased in to the expanded configuration. In such an alternative embodiment, the joint portions are not present, as the material and formation of the implant biases the implant into the expanded configuration.

With the implant 922 disposed within the sheath 944, a distal end 998 of the sheath 994 can be inserted percutaneously into a spinal column, for example, in a direction of arrow A shown in FIG. 21. The distal end 998 of the sheath 944 can be positioned adjacent a spinous process S. An insertion tool 996 (shown in FIG. 20) can be movably disposable within and/or relative to the sheath 944 and used to move the implant 922 distally and outside of the sheath 944. As the implant 922 is moved to a location outside of the sheath 944, the sheath 944 can be moved proximally such that the implant 922 can assume its biased configuration and wrap partially around the spinous process S and the sheath 944 can be removed from the body. In some embodiments, the sheath 944 and the insertion tool 996 can be a curved shape and/or can be configured to position the implant 922 between adjacent spinous processes via a path from a side of the adjacent spinous processes.

The implant 922 can be configured to be coupled to the spinous process using any of the various coupling methods described above for other embodiments. In addition, more than one implant 922 can be used as described above, for example, on adjacent spinous processes. In alternative embodiments, the joints 990 and 992 can each include a fold or break-away line, or other type of hinge instead of a biasing member. In such an embodiment, the first portion 984 can be folded or pivoted with respect to the second portion 986, and the third portion 988 can folded or pivoted with respect to the second portion 986.

FIG. 22 illustrates an embodiment of an implant that is similar to the embodiment of FIGS. 20 and 21. In this embodiment, an implant 1022 includes a first portion 1084 coupled to a second portion 1086 at a joint 1090. The joint 1090 can include a biasing member as described above. The implant 1022 can be coupled to a spinous process S in a similar manner as described above. For example, the implant 1022 can be inserted into a sheath or cannula (not shown), and the sheath and implant 1022 can then be percutaneously inserted into a spinal column, for example, in a direction of arrow B shown in FIG. 22. The implant 1022 can alternatively be inserted from an opposite side of the spinous process. An insertion tool (not shown) can move the implant 1022 outside of the sheath allowing the implant 1022 to assume its expanded configuration and wrap partially around the spinous process. As with the previous embodiment, the implant 1022 can be coupled to the spinous process using any of the coupling methods as described above. The implant 1022 also includes a lip portion 1099 that can help maintain the position of the implant 1022 relative to the spinous process S.

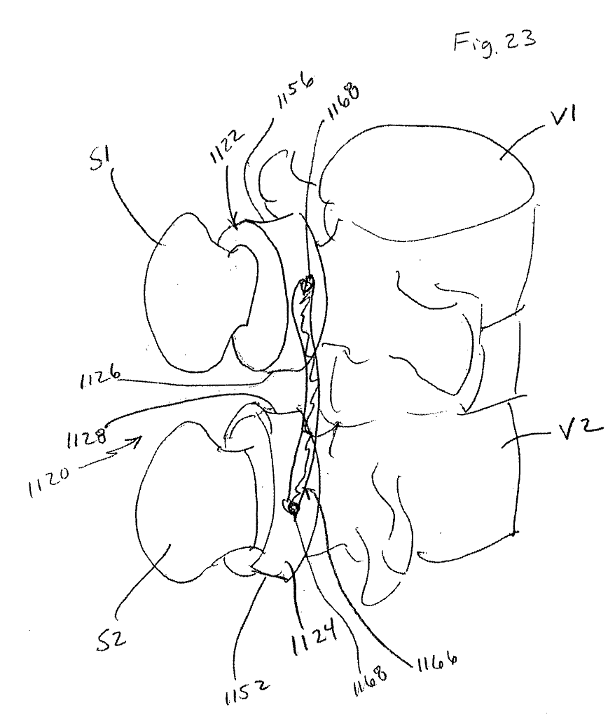

FIG. 23 illustrates a pair of implants according to another embodiment shown coupled to adjacent spinous processes. A medical device 1120 includes a first implant 1122 and a second implant 1124. The first implant 1122 is coupled to a spinous process S1 of a vertebra V1, and the second implant 1124 is coupled to a spinous process S2 of a second vertebra V2. The first implant 1122 includes an outer surface 1126 that can contact an outer surface 1128 of the second implant 1124. The first implant 1122 also includes an outer surface 1156 that can contact an outer surface of another implant (optional, not shown) that can be coupled to a spinous process of a superior vertebra (not shown). The second implant 1124 also includes an outer surface 1152 that can contact an outer surface of another implant (optional, not shown) that can be coupled to a spinous process of an inferior vertebra (not shown). As described previously, the outer surfaces 1126 and 1128 of the implants 1122 and 1124, respectively, can contact each other when the spinal column is in extension and can be out of contact with each other such that they are at a spaced distance from each other when the spinal column is in flexion.

The first implant 1122 and the second implant 1124 can each be secured to the respective spinous process with a variety of different coupling methods as described herein, such as with adhesives, screws, pins, nails, protrusions, crimping, or a friction fit. The same coupling methods or different coupling methods can be used for each implant. Although the first implant 1122 and second implant 1124 are each shown having the same configuration, other combinations of implants can be used.

In this embodiment, the medical device 1120 also includes a linking member 1166 to couple the first implant 1122 to the second implant 1124. As shown in FIG. 23, a first end of the linking member 1166 can be coupled to a side wall of the first implant 1122 and a second end of the linking member 1166 can be coupled to a side wall of the second implant 1124. The linking member 1166 can be, for example a rope, a cable, a tether, a chord, etc. The linking member 1166 can be formed with various materials, such as various metals or plastics, and can be substantially rigid or stiff, flexible, or include portions that are flexible and portions that are rigid. The linking member 1166 can be secured to the implant 1122 and implant 1124 with various couplers 1168, such as for example, a tack, pin, screw, nail, or clip. In some embodiments, the ends of the linking member 1166 can be pushed through an opening through a side wall of the implants and held with a friction fit.

As stated above, during movement of the spinal column, the outer surfaces of the first implant 1122 and second implant 1124 can be in an out of contact with each other and/or with another implant coupled to an adjacent spinous process. The implants 1122 and 1124 can be used to limit extension, when, for example, the outer surface 1126 contacts the outer surface 1126. The implants 1122 and 1124, together with the linking member 1166 can also be used to limit flexion during movement of the spinal column. For example, as the spinous processes S1 and S2 move apart from each other during flexion of the spinal column, the linking member 1166 can limit how far apart the spinous processes S1 and S2 can move. Although one linking member 1166 is shown in FIG. 23, in other embodiments, a medical device can include two linking members. For example, a linking member can be coupled along one side of adjacent spinous processes as shown in FIG. 23, and a second linking member can be coupled to the implants on the other side of the spinous processes. In some embodiments, a device (not shown) such as an interspinous-process implant, can be placed between the outer surface 1126 and the outer surface 1128 as described above for previous embodiments.

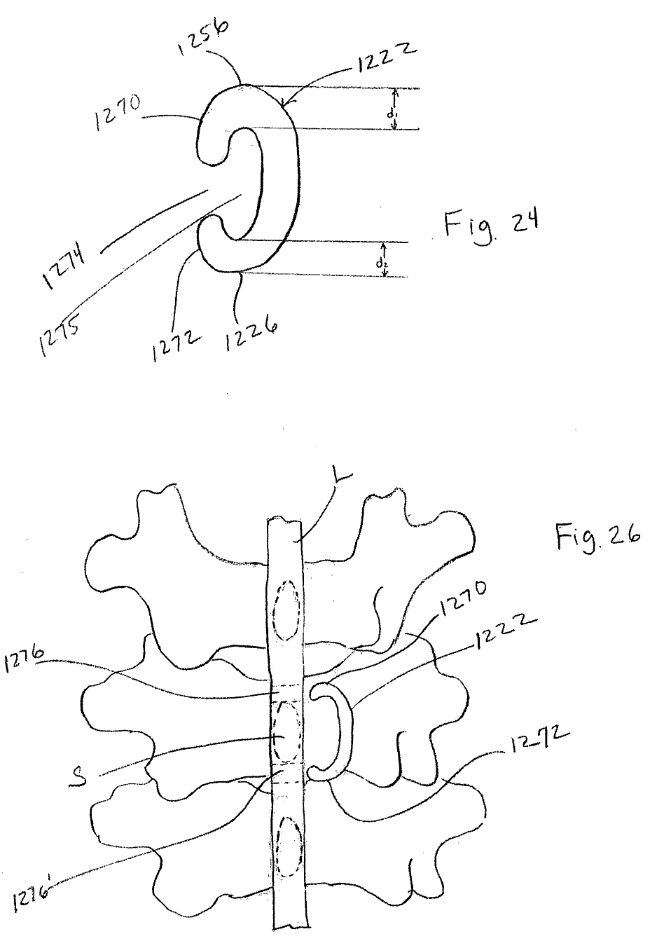

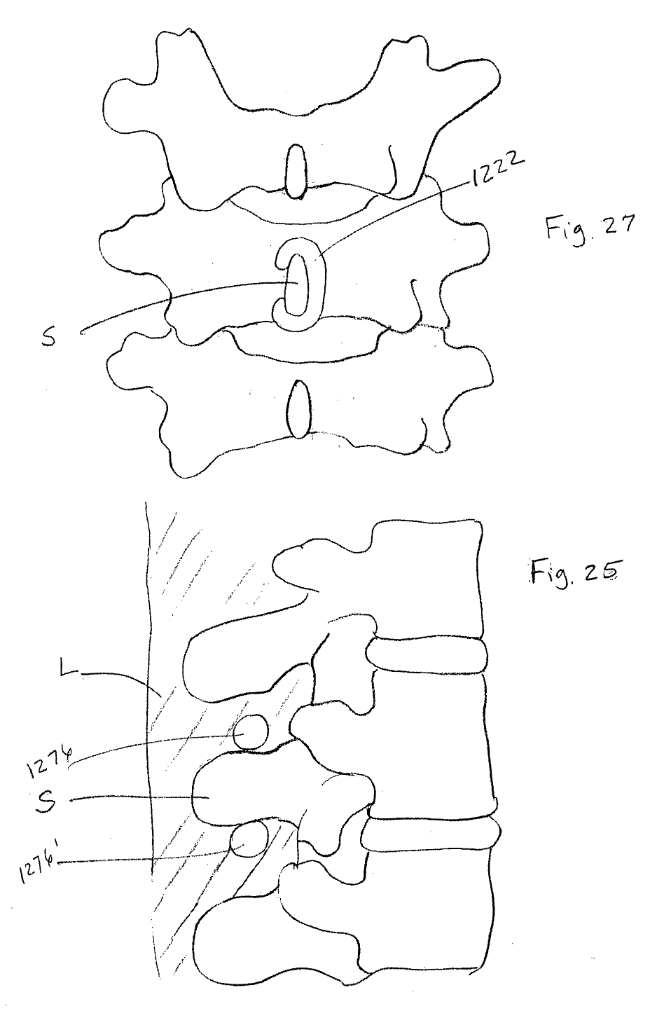

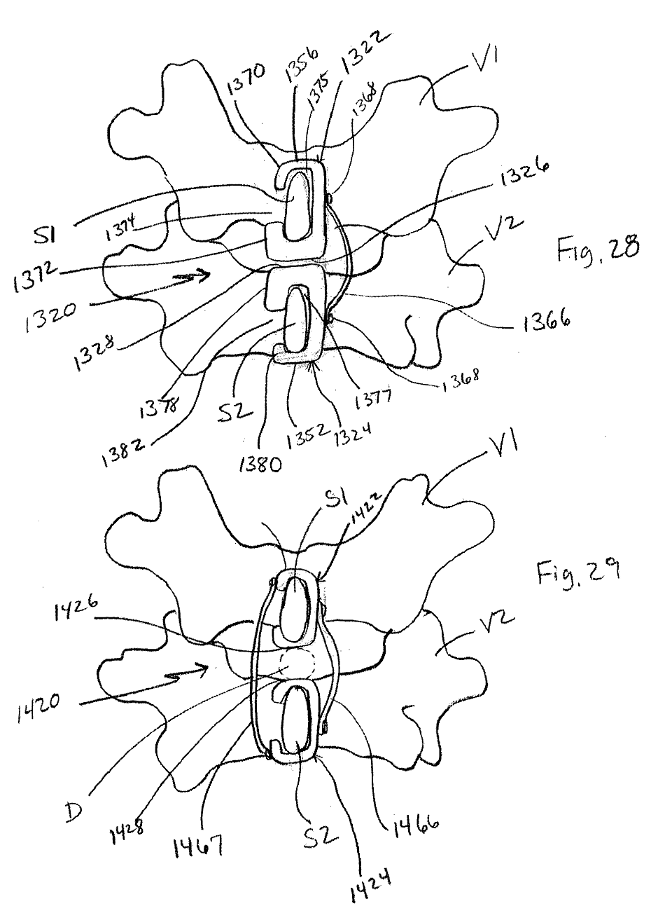

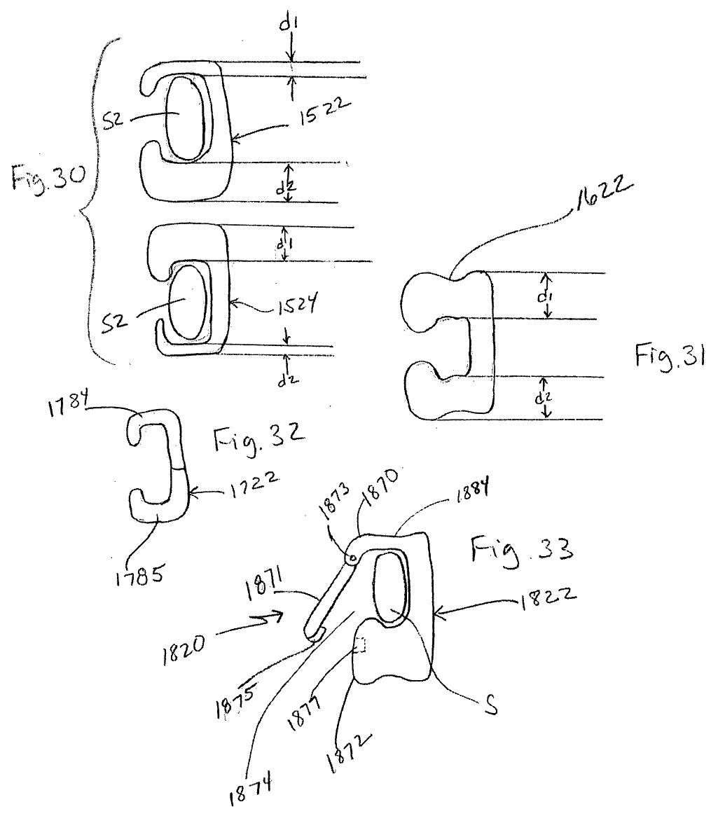

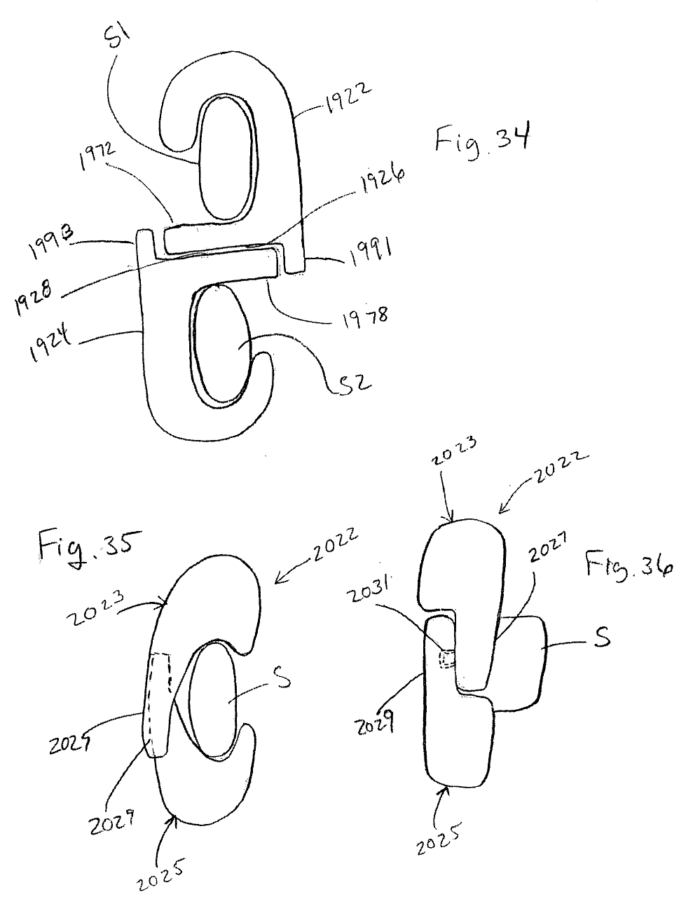

FIG. 24 is a rear view (i.e., a posterior view when disposed within a body) that illustrates an embodiment of an implant that is substantially C-shaped. Such an implant can be inserted into a spinal column and coupled to a spinous process by a lateral (e.g., side) approach. An implant 1222 can be coupled to a spinous process (not shown in FIG. 24) and includes an outer surface 1226 and an outer surface 1256. The outer surfaces 1226 and 1256 can be in and out of contact with another optional implant coupled to an adjacent spinous process as described above. The outer surfaces 1226 and 1228 can also be configured to contact a device or spacer, such as an interspinous-process implant, disposed between two adjacent spinous processes as described above.

The implant 1222 also includes a first arm 1270 and a second arm 1272. Each arm 1270 and 1272 having an end that collectively define an opening 1274. The opening 1274 is in communication with an interior region 1275 defined by the implant 1222. The interior region 1275 can receive at least a portion of a spinous process therein when the implant 1222 is coupled to the spinous process (described in more detail below). The various portions (e.g., the arms 1270 and 1272) of the implant 1222 can be a variety of different shapes and sizes. For example, the implant 1222 can have a dimension d1 and a dimension d2 as shown in FIG. 24 that is substantially the same, or d1 can be different than d2. In some embodiments, the dimension d1 is greater than the dimension d2, while in other embodiments, the dimension d2 is greater than the dimension d1. Various embodiments illustrating the different combinations of the dimensions d1 and 2 are described below.

As stated above, the C-shape of the implant 1222 allows the implant 1222 to be placed on or coupled to a spinous process from a lateral (e.g., side) direction. By placing the implant 1222 from the side, the amount of the spinal ligaments (e.g., the superspinous ligament and the interspinous ligament) that will need to be cut (e.g., resect) to insert the implant can be reduced. A procedure to insert the implant 1222 includes cutting a small incision in the subject body at the midline of the back or to the right or left of the midline, moving the tissue and performing a dissection to gain access to the spinal column. As shown in FIG. 25, a hole 1276 can be cut in the interspinous ligament L, as shown in FIG. 25, above the target spinous process S to which the implant is to be coupled. A second hole 1276′ in the interspinous ligament can be cut below the target spinous process S.

The implant 1222 (or other substantially C-shaped implant) can be inserted through the incision in the subject body's back, and positioned adjacent a side of the spinous process S with the first arm 1270 and the second arm 1272 positioned such that they can be placed through the openings 1276 and 1276′, respectively, as shown in FIG. 26. The arms 1270 and 1272 can be flexible or deformable to assist with placing them through the openings 1276, 1276′. FIG. 27 illustrates the implant 1222 coupled to the spinous process S. Although only one implant is shown with respect to FIGS. 25-27, it should be understood that additional implants can be coupled to spinous processes adjacent to the spinous process S as described herein.

FIG. 28 illustrates an embodiment of a medical device including a pair of substantially C-shaped implants and a linking member. A medical device 1320 includes a first implant 1322 and a second implant 1324. The first implant 1322 is coupled to a spinous process S1 of a vertebra V1, and the second implant 1324 is coupled to a spinous process S2 of a second vertebra V2. The first implant 1322 includes an outer surface 1326 that can contact an outer surface 1328 of the second implant 1324. The first implant 1322 also includes an outer surface 1356 that can contact an outer surface of another implant (optional, not shown) that can be coupled to a spinous process of a superior vertebra (not shown). The second implant 1324 also includes an outer surface 1352 that can contact an outer surface of another implant (optional, not shown) that can be coupled to a spinous process of an inferior vertebra (not shown). As described previously, the outer surfaces 1326 and 1328 of the implants 1322 and 1324, respectively, can contact each other when the spinal column is in extension and can be out of contact with each other such that they are at a spaced distance from each other when the spinal column is in flexion. The surfaces 1326, 1328, 1352, 1356 can each also be configured to contact a device, such as an interspinous process implant or spacer, as described previously.

The implant 1322 also includes an arm 1370 and an arm 1372, and the second implant 1324 includes an arm 1378 and an arm 1380. As with the embodiment of FIG. 23, the arm 1370 and the arm 1372 each includes an end that collectively define an opening 1374 in communication with an interior region 1375. Likewise, the arm 1378 and the arm 1380 each includes an end that collectively define an opening 1382 in communication with an interior region 1377. The implant 1322 and the implant 1324 can each be placed on or coupled to the respective spinous processes S1 and S2 in the same manner as described above for implant 1222.

In this embodiment, the medical device 1320 also includes a linking member 1366 to couple the first implant 1322 to the second implant 1324. As with the embodiment of FIG. 23, a first end of the linking member 1366 can be coupled to a side wall of the first implant 1322 and a second end of the linking member 1366 can be coupled to a side wall of the second implant 1324. The linking member 1366 can be a variety of different configurations and be coupled to the implant 1322 and implant 1324 with various couplers 1368, as previously described.

A portion of the implant 1322 below the spinous process S1 is thicker than a portion of the implant 1322 above the spinous process S1. Similarly, a portion of the implant 1324 above the spinous process S2 is thicker than a portion of the implant 1324 below the spinous process S2. This configuration of the implant 1322 and 1324 allows the outer surfaces of the first implant 1322 and second implant 1324 to move in an out of contact with each other during movement of the spinal column. For example, the implants 1322 and 1324 can limit extension of the spinal column when the outer surface 1326 contacts the outer surface 1328. The implants 1322 and 1324, together with the linking member 1366, can also limit flexion during movement of the spinal column. For example, as the spinous processes S1 and S2 move apart from each other during flexion of the spinal column, the linking member 1366 can limit how far apart the spinous processes S1 and S2 can move.

FIG. 29 illustrates an embodiment of a medical device having a pair of substantially C-shaped implants and two linking members. A medical device 1420 includes a first implant 1422 and a second implant 1424. The first implant 1422 is coupled to a spinous process S1 of a vertebra V1, and the second implant 1424 is coupled to a spinous process S2 of a second vertebra V2. The implants 1422 and 1424 are similarly formed as the previous embodiment, except in this embodiment, a portion of the implant 1422 above the spinous process S1 and a portion of the implant 1422 below the spinous process S1 are substantially the same thickness. The second implant 1424 is similarly formed. Thus, in this embodiment, the first implant 1422 includes an outer surface 1426 that can contact a portion of a device D (e.g., an interspinous-process implant or spacer) and the second implant 1424 includes an outer surface 1428 that contact a different portion of the device D. The implants 1422 and 1424 can be coupled to the respective spinous processes S1 and S2 in the same manner as previously described for substantially C-shaped implants.

In this embodiment, the medical device 1420 also includes a first linking member 1466 and a second linking member 1467. The first linking member 1466 and the second linking member 1467 can each be configured similar as described for previous embodiments of a linking member and be coupled to the first implant 1422 and second implant 1424 in the same manner. As with the previous embodiment, the implants 1422 and 1424 together with the linking members 1466 and 1467 can limit flexion of the spinal column. The implants 1422 and 1424 can also assist in limiting extension of the spinal column when the outer surfaces 1426 and 1428 contact the device D.

FIGS. 30-40 illustrate various different embodiments of an implant, each having a substantially C-shape and that can be coupled to a spinous process in the same manner as described above for other C-shaped implants. Each of the implants described below also include outer surfaces that can either be in and out of contact with an outer surface of another implant or be in and out of contact with a device placed adjacent the implant as previously described.

FIG. 30 illustrates a first implant 1522 shown coupled to a spinous process S1 and a second implant 1524 shown coupled to a spinous process S2. The implant 1522 has an upper portion having a dimension d1 that is smaller than a dimension d2 of a lower portion of the implant 1522. The implant 1524 has an upper portion with a dimension d1 that is greater than a dimension d2 of a lower portion. The implants 1522 and 1524 are configured similar to the implants of FIG. 28. FIG. 31 illustrates an implant 1622 having a dimension d1 that is substantially the same as a dimension d2.

FIG. 32 illustrates an embodiment of an implant formed with two different materials. An implant 1722 includes a first portion 1784 and a second portion 1784. Each of the first portion 1784 and the second portion 1785 can be formed with a different material. For example, the first portion 1784 can be formed with a metal such as, for example, a rigid material, and the second portion 1785 can be formed with a flexible material. In some embodiments, the first portion 1784 is formed with a metallic material and the second portion 1785 is formed with a plastic material. These are merely examples of the types of materials that can be used, as other combinations of materials can alternatively be used.

FIG. 33 illustrates an embodiment of a medical device having an implant and a closure member. A medical device 1820 includes an implant 1822 and a closure member 1871 that is pivotally coupled to a first arm 1870 of the implant 1822 at a pivot location 1873. In this embodiment, the closure member 1871 is a substantially rigid component. In alternative embodiments, the closure member 1871 can be flexible. For example, the closure member 1871 can be formed with rope, cable, cord, fabric, metal, plastic, rubber etc. The closure member 1871 includes a coupler 1875 that can be matingly received within an opening 1877 in a second arm 1872. For example, the coupler 1875 and the opening 1877 can be configured to provide a snap-fit connection. In alternative embodiments, the closure member 1871 can be pivotally coupled to the arm 1872 and the coupler 1875 can be configured to be received within an opening on the arm 1870.

In this embodiment, the implant 1822 can be placed on a spinous process S in a manner similar to the procedure described above for implant 1222. For example, the closure member 1871 can be placed in an open position such that it is substantially parallel to a portion 1884 of the arm 1873 of the implant 1822. The closure member 1871 and arm 1873 can then be placed through an opening in an interspinous ligament above the spinous process S as described with respect to FIG. 25. The arm 1872 can likewise be placed through an opening below the spinous process. The closure member 1871 can then be moved to a closed position with the coupler 1875 disposed within the mating opening 1877 such that an opening 1874 defined by the implant 1822 is closed-off. Although the implant 1822 can maintain its position on the spinous process S without the closure member 1871, the closure member 1871 can provide further securement of the implant 1822 on the spinous process S.

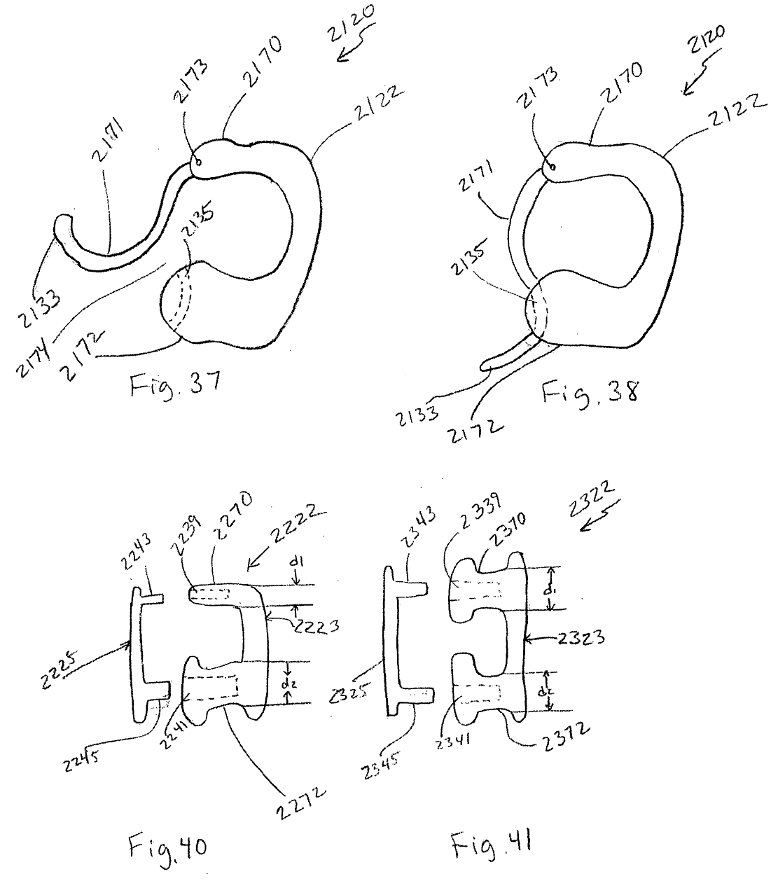

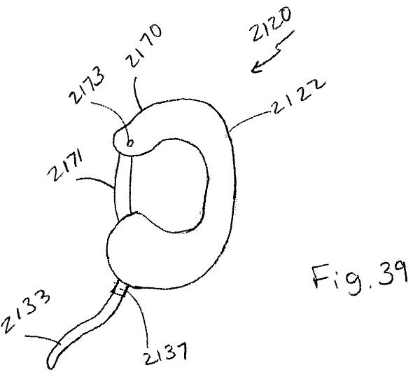

FIG. 34 illustrates a pair of implants that can matingly engage each other when coupled to adjacent spinous processes. An implant 1922 is shown coupled to a spinous process S1 and an implant 1924 is shown coupled to a spinous process S2. The implant 1922 includes an arm 1972 and a stop portion 1991. The second implant 1924 includes an arm 1978 and a stop portion 1993. An outer surface 1926 of the first implant 1922 can be in and out of contact with an outer surface 1928 of the second implant during movement of a spinal column. The implants The arms 1972 and 1978 can move laterally with respect to each other but are limited by the stop portions 1991 and 1993 in the lateral direction.

FIGS. 35 and 36 illustrate a two-part implant. An implant 2022 includes a first portion 2023 and a second portion 2025. FIG. 35 illustrates a rear view of the implant 2022 shown coupled to a spinous process S, and FIG. 36 illustrates a side view of the implant 2022 and spinous process S. The first portion 2023 and the second portion 2025 can be placed on the spinous process S with a lateral or side approach as described above. The first portion 2023 includes an arm 2027 and the second portion 2025 includes an arm 2029. After placing the first portion 2023 and the second portion 2025 on the spinous process S, the arm 2027 can be coupled to the arm 2029 with, for example, a snap-fit coupling 2031. Other coupling methods can alternatively be used, for example, a screw can be placed through the arm 2027 and the arm 2029. In some embodiments, a key-way and lock mechanism can be used to slidingly interlock the first portion 2023 with the second portion 2025.

FIGS. 37-39 illustrate another embodiment of a medical device that includes a closure member coupled to an implant. A medical device 2120 includes an implant 2122 and a closure member 2171. The closure member 2171 is coupled to a first arm 2170 at a location 2173 with for example a pin, nail, tack, or other coupling device. In this embodiment, the closure member 2171 is in the form of a flexible strap, but as described above for closure member 1871, the closure member 2171 can be formed with a variety of different materials, such as, for example, rope, cable, chord, metal, plastic, etc., and can be flexible or rigid.

The implant 2122 can be placed on a spinous process in a similar manner as described for implant 1822. For example, the closure member 2171 can be moved to a position such that it is substantially parallel with the arm 2170, and the arm 2170 and closure member 2171 can be inserted through an opening cut in a ligament above the spinous process. An arm 2172 can likewise be inserted through an opening in the ligament below the spinous process.

After the implant is placed on the spinous process, the closure member 2171 can be moved to a closed position to close-off an opening 2174 defined by the implant 2122. Specifically, an end 2133 of the closure member 2171 can be received through an opening 2135 in the second arm 2172 of the implant 2122 as shown in FIG. 38. In this embodiment, the implant is formed with a flexible or deformable material, and the closure member 2171 can be pulled through the opening 2135 such that the implant 2122 at least partially bends or deforms around the spinous process, as shown in FIG. 39. A clip 2137 or other stop member can be coupled to the closure member 2171 to prevent the end 2133 from slipping back through the opening 2135. In some embodiments, the implant can alternatively define teeth (not shown) on an edge of the opening 2135 that allow the closure member 2171 to be pulled through the opening 2137 in only one direction. For example, the teeth can be angled such that the closure member 2171 can be pulled through the opening 2135 in a first direction, but engage the closure member 2171 in an opposite direction, which prevents the closure member 2171 from backing out of the opening 2135.

FIG. 40 illustrates another embodiment of a two-piece implant that can surround a portion of a spinous process. An implant 2222 includes a first portion 2223 and a second portion 2225. In this embodiment, a first arm 2270 of the first portion 2223 has a dimension d1 that is smaller than a dimension d2 of a second arm 2272 of the first portion 2223. The first portion 2223 of the implant 2222 defines a first opening 2239 in the first arm 2270, and a second opening 2241 in the second arm 2272. The second portion 2225 of the implant 2222 includes a first coupler 2243 and a second coupler 2245 that can be received within the openings 2239 and 2241, respectively. The couplers 2243 and 2245 can be, for example, configured to provide a snap-fit connection with the openings 2239 and 2241, respectively.

To couple the implant 2222 to a spinous process, the first portion 2223 of the implant 2222 can be placed on a spinous process in the same manner as described above for other C-shaped implants, for example, with reference to FIG. 25. In this embodiment, the openings in the interspinous ligament can extend through the interspinous ligament to an opposite side of the interspinous ligament. With the arms 2270 and 2272 of the first portion 2223 placed through the openings in the interspinous ligament, the second portion 2225 can be positioned on an opposite side of the spinous process and coupled to the first portion 2223. For example, in some embodiments, the arms 2270 and 2272 of the first portion 2223 extend through the openings to the opposite side of the interspinous ligament such that the couplers 2243 and 2245 of the second portion 2225 do not get placed into the openings when being coupled to the arms 2270 and 2272 of the first portion 2223. In other embodiments, the arms 2270 and 2272 of the first portion 2223 do not extend through the openings to an opposite side of the interspinous ligament. In such an embodiment, the couplers 2243 and 2245 on the second portion 2225 can be placed through the openings of the interspinous ligament (from the opposite side of the ligament) to be coupled to the arms 2270 and 2272.

FIG. 41 illustrates an implant that is similar to the implant 2222. An implant 2322 includes a first portion 2323, a second portion 2325, a first arm 2370, a second arm 2372, openings 2339 and 2341 and couplers 2343 and 2345. The first portion 2323 and the second portion 2325 can be coupled to a spinous process in the same manner as described above for implant 2322. In this embodiment, the first arm 2370 has a dimension d1 that is substantially the same as a dimension d2 of the second arm 2372.

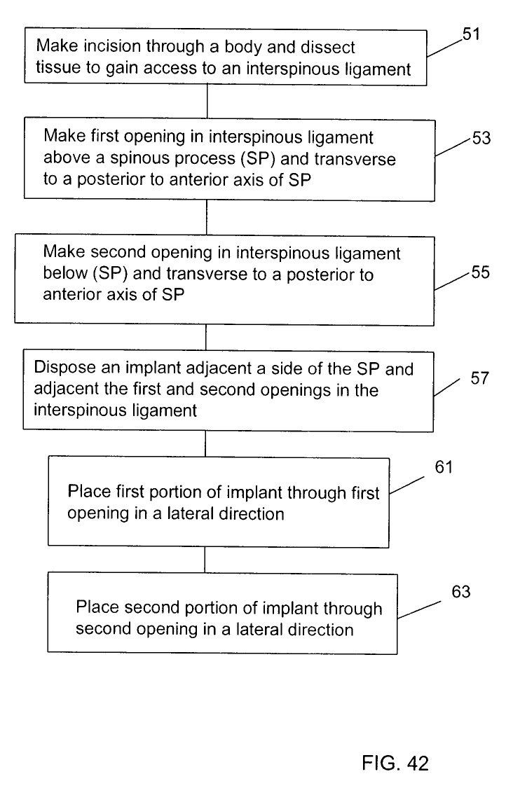

FIG. 42 is a flowchart illustrating a method of placing an implant on a spinous process according to an embodiment of the invention. At 51, an incision is made through a body and a dissection can be performed on the tissue to gain access to an interspinous ligament. At 53, a first opening is made in the interspinous ligament at a location above the spinous process transverse to a posterior-to-anterior axis of the spinous process. At 55, a second opening is made in the interspinous ligament at a location below the spinous process and transverse to a posterior to anterior axis of the spinous process. At 57, an implant is disposed adjacent (e.g., laterally inserted to) a side of a spinous process and adjacent the first and second openings in the interspinous ligament. The implant can be, for example, substantially C-shaped as described above. At 59, a first portion of the implant is placed through the first opening over a top side of the spinous process in a lateral direction (i.e., transverse from a posterior-to-anterior axis of the spinous process). At 61, a second portion of the implant is placed under a bottom side of the spinous process in a lateral direction (i.e., transverse from a posterior-to-anterior axis of the spinous process) such that at least a portion of the spinous process is disposed within an interior region defined by the implant.

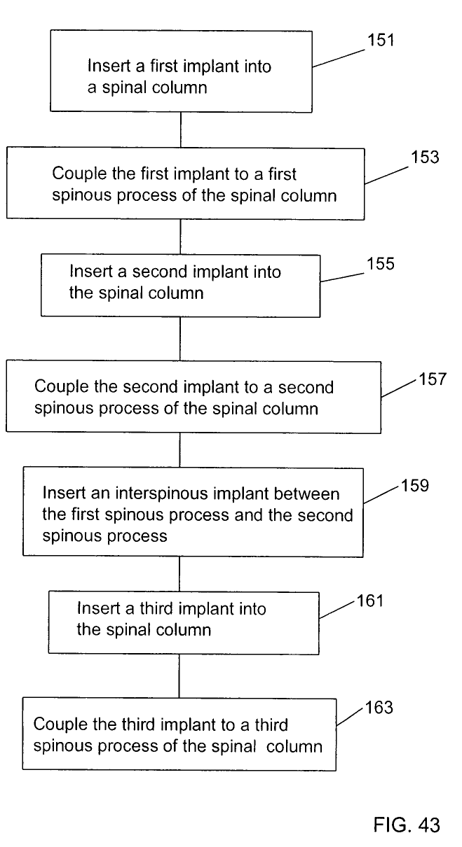

FIG. 43 is a flowchart illustrating another method according to an embodiment of the invention. At 151, a first implant is inserted into a spinal column. The first implant can optionally be percutaneously inserted into the spinal column. At 153, the first implant is coupled to a first spinous process as described herein. At 155, a second implant is inserted into the spinal column. The second implant can optionally be inserted percutaneously into the spinal column. At 157 the second implant is coupled to an adjacent second spinous process as described herein. In some embodiments, an interspinous-process implant is optionally disposed between the first spinous process and the second spinous process, either percutaneously or otherwise, at 159. The first implant and the second implant each include an outer surface that can move in and out of contact with either each other or a device disposed between the adjacent spinous processes. At 161, a third implant can optionally be inserted (e.g., percutaneously) into the spinal column. At 163, the third implant can optionally be coupled to a third spinous process as described herein. The third spinous process is adjacent the first spinous process or the second spinous process. The third implant includes an outer surface that can contact a device disposed between the third spinous process and either the first spinous process or the second spinous process. In other embodiments, the third implant can contact a portion of an outer surface of either the first implant or the second implant.

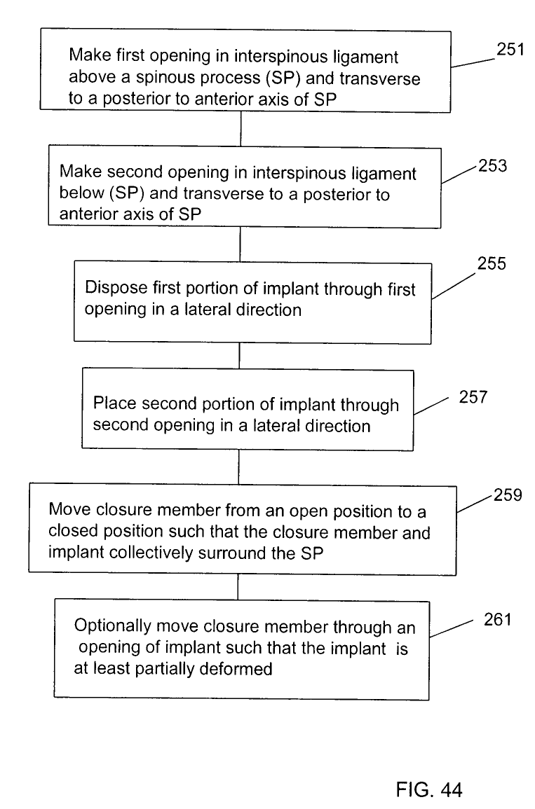

FIG. 44 is a flowchart illustrating another method of inserting and placing an implant on a spinous process. At 251, a first opening is made in the interspinous ligament at a location above the spinous process and transverse to a posterior-to-anterior axis of the spinous process. At 253, a second opening is made in the interspinous ligament at a location below the spinous process and transverse to a posterior to anterior axis of the spinous process. The first and second openings each extend through the interspinous ligament to an opposite side of the spinous process. At 255, a first portion of an implant is disposed through the first opening (e.g., via a lateral direction). At 257 a second portion of the implant is disposed through the second opening (e.g., via a lateral direction). At 259 a closure member coupled to the implant is moved from an open position to a closed position such that the closure member and the implant collectively surround the spinous process. At 261, the closure member can optionally be moved through an opening of the implant such that the implant is at least partially deformed.

The implants for any of the embodiments can be formed with any suitable material used for such medical devices. For example, the implants can each be formed with biocompatible metal materials, such as stainless steel, titanium, titanium alloy, surgical steel, metal alloys, or suitable biocompatible plastic materials, such as various polymers, polyetheretherketone (PEEK), carbon fiber, ultra-high molecular weight (UHMW) polyethylene, etc., or various elastic materials, flexible materials, various rubber materials, or combinations of various materials thereof. In addition, any of the embodiments of an implant can be formed with one or more compliant materials. An implant can also be formed with a shape-memory material or can be formed such that the implant can be heat set into a biased configuration.

While various embodiments of the invention have been described above, it should be understood that they have been presented by way of example only, and not limitation. Where methods and steps described above indicate certain events occurring in certain order, those of ordinary skill in the art having the benefit of this disclosure would recognize that the ordering of certain steps may be modified and that such modifications are in accordance with the variations of the invention. Additionally, certain of the steps may be performed concurrently in a parallel process when possible, as well as performed sequentially as described above. The embodiments have been particularly shown and described, but it will be understood that various changes in form and details may be made.

For example, although various embodiments have been described as having particular features and/or combinations of components, other embodiments are possible having a combination or sub-combination of any features and/or components from any of embodiments as discussed above. For example, any of the various embodiments of an implant can be configured to be secured to a spinous process using any of the described coupling methods. The various embodiments of an implant can have other shapes, sizes and configurations than those specifically described. For example, components such as a linking member or a closure member can be included in any of the embodiments. Any of the embodiments of an implant can be sized (e.g., a thickness extending between adjacent spinous process and/or adjacent implants) to at least partially limit flexion and/or at least partially limit extension between adjacent spinous processes. Any of the embodiments of an implant can be formed with one or more materials and can be flexible, rigid or both.

An implant can be symmetrical, such as, for example, the implant 122 or the implant 222, or an implant can be non-symmetrical, such as for example, the implant 1022. In addition, although the embodiments above are primarily described as being spinal implants configured to be coupled to a spinous process, in alternative embodiments, the implants are configured to be coupled to other bone, tissue or other bodily structure where it is desirable to protect the particular anatomy from wear caused by another device or implant disposed therein.

Any of the various embodiments of an implant can be configured such that during extension of the spinal column, the implant is in contact with, or has a spaced distance to, either another implant coupled to an adjacent spinous process or to an interspinous-process implant. Similarly, any of the embodiments of an implant can be configured such that during flexion of the spinal column, the implant is in contact with, or is at a spaced distance from, either another implant coupled to an adjacent spinous process or to an interspinous-process implant. In some embodiments, an implant remains in contact with another implant or an interspinous-process implant during both flexion and extension. In other embodiments, an implant is at a spaced distance from another implant or an interspinous-process implant during both flexion and extension.

Various combinations of the different embodiments of an implant can be implanted within a spinal column. For example, a procedure can include coupling one implant to one spinous process, or can include two or more implants each being coupled to a different spinous process within a spinal column. In such a case, the two or more implants can have the same or different configurations. For example, one or more implants can be used that are configured to be coupled to only one side (e.g., inferior or superior) of a spinous process and/or one or more implants can be used that are configured to surround a spinous process.

Methods for insertion and placement of the various embodiments of an implant can also vary. For example, the arms of a C-shaped implant can be placed on a spinous process sequentially in any order (e.g., an upper arm can be inserted before or after a lower arm), or at substantially the same time. In addition, although a lateral or side approach is described, a posterior approach to couple or place an implant on a spinous process can alternatively be used. In some embodiments, the implants can be inserted percutaneously into a spinal column. For example, an implant can be formed with a shape-memory material such that it can be substantially straightened within an insertion cannula or sleeve, and will assume an expanded or biased configuration for placement on a spinous process. In such alternative embodiments, an implant formed with a shape-memory material can be percutaneously inserted through a cannula.

What is claimed is:

1. A method, comprising:
disposing an implant adjacent a side of a spinous process;
placing a first portion of the implant over a top side of the spinous process in a lateral direction; and
placing a second portion of the implant under a bottom side of the spinous process in a lateral direction such that at least a portion of the spinous process is disposed within an interior region defined by the implant.

2. The method of claim 1, wherein the implant is substantially C-shaped.

3. The method of claim 1, wherein the implant is a first implant, the spinous process is a first spinous process, the method further comprising:
coupling a second implant to a second spinous process adjacent the first spinous process

4. The method of claim 1, wherein the spinous process is a first spinous process, the method further comprising:
placing an interspinous-process spacer adjacent the implant between the first spinous process and an adjacent second spinous process.

5. The method of claim 1, wherein the spinous process is a first spinous process, the method further comprising:
placing an interspinous-process spacer adjacent the implant between the first spinous process and an adjacent second spinous process,
an outer surface of the implant configured to be at a spaced distance from the spacer when the spinal column is in flexion.

6. The method of claim 1, wherein the implant includes a closure member, the method further comprising:
moving the closure member from an open position to a closed position in which the implant surrounds a portion of the spinous process.

7. The method of claim 1, further comprising:
cutting a first opening in an interspinous ligament at a location above the spinous process, the first opening being transverse to a posterior to anterior axis of the spinous process; and
cutting a second opening in the interspinous ligament at a location below the spinous process, the second opening being transverse to a posterior to anterior axis of the spinous process.

8. The method of claim 1, wherein the placing the first portion and the placing the second portion is substantially simultaneously.

9. The method of claim 1, wherein the placing the first portion and the placing the second portion is sequential.

10. An apparatus, comprising:
a first implant configured to be coupled to a first spinous process of a spinal column;
a second implant configured to be coupled to a second spinous process of the spinal column; and
a linking member coupled to the first implant and the second implant,
a surface of the first implant configured to contact at least one of the second implant or an interspinous-process implant when the spinal column is in extension and be at a spaced distance from the at least one of the second implant or the interspinous process implant when the spinal column is in flexion,
the linking member configured to at least partially limit a space between the first spinous process and the second spinous process when the spinal column is in flexion.

11. The apparatus of claim 10, wherein a surface of the second implant configured to contact at least one of the first implant and the interspinous process implant when the spinal column is in extension.

12. The apparatus of claim 10, wherein the surface of the first implant is spaced apart from the at least one of the second implant or the interspinous process implant when the spinal column is in flexion.

13. The apparatus of claim 10, wherein a surface of the second implant is spaced apart from the first implant or the interspinous process implant when the spinal column is in flexion.

14. The apparatus of claim 10, wherein the first implant includes a coupling portion configured to couple the first implant to the first spinous process.

15. The apparatus of claim 10, wherein the second implant includes a coupling portion configured to couple the second implant to the second spinous process.

16. The apparatus of claim 10, wherein the linking member is fixedly coupled to at least one of the first implant or the second implant.

17. The apparatus of claim 10, wherein the linking member is substantially rigid,

18. The apparatus of claim 10, wherein the linking member is substantially flexible.

19. An apparatus, comprising:
a first implant configured to be coupled to a spinous process of a spinal column, the implant configured to cover a posterior end of the spinous process, a surface of the first implant configured to contact at least one of a second implant or an interspinous-process implant when the spinal column is in extension and be at a spaced distance from the at least one of the second implant or the interspinous-process implant when the spinal column is in flexion.

20. The apparatus of claim 19, wherein the implant is a first implant, the spinous process is a first spinous process, the apparatus further comprising:
a second implant configured to be coupled to a second spinous process adjacent the first spinous process, the first implant being separate from the second implant.

21. The apparatus of claim 19, the implant is a first implant, the spinous process is a first spinous process, the apparatus further comprising:
a second implant configured to be coupled to a second spinous process adjacent the first spinous process; and
a spacer configured to be disposed between the first implant and the second implant, the second implant having an outer surface portion configured to be removably in contact with the spacer.

22. The apparatus of claim 19, wherein the spinous process is a first spinous process, the entire implant is disposed at a spaced distance from both a second spinous process superior to the first spinous process and a third spinous process inferior to the first spinous process.

23. The apparatus of claim 19, wherein the implant has an inner surface configured to couple the implant to the spinous process.

24. The apparatus of claim 19, wherein the first implant includes a portion configured to couple the implant to the spinous process.

25. The apparatus of claim 19, wherein the spinous process is a first spinous process, when the implant is coupled to the first spinous process the implant can move with the first spinous process independent from movement of a second spinous process superior to the first spinous process and a third spinous process inferior to the first spinous process.

26. An apparatus, comprising:
an implant configured to be coupled to a spinous process of a spinal column, the implant having an outer surface configured to contact at least one of a second implant or an interspinous-process spacer; and
a closure member coupled to the implant, the closure member having an open configuration to place the implant on the spinous process and a closed configuration to secure the implant to the spinous process.

27. The apparatus of claim 26, wherein the entire implant is disposed at a spaced distance from an adjacent spinous process when the implant is coupled to the spinous process.

28. The apparatus of claim 26, wherein the spinous process is a first spinous process, the implant is a first implant, the apparatus further comprising:
a second implant configured to be coupled to a second spinous process adjacent the first spinous process.

29. The apparatus of claim 26, wherein the closure member is pivotally coupled to the implant.

30. The apparatus of claim 26, wherein the implant is a first implant, the spinous process is a first spinous process, the apparatus further comprising:
a second implant coupled to a second spinous process, the outer surface of the first implant is at a spaced distance from an outer surface of the second implant when the spinal column is in flexion.

31. The apparatus of claim 26, wherein a portion of the closure member is configured to be received through an opening defined by the implant, the closure member configured to at least partially bend the implant around the spinous process when the portion of the closure member is pulled through the opening.

32. The apparatus of claim 26, wherein the implant is a first implant, the spinous process is a first spinous process, the apparatus further comprising:
a second implant configured to be coupled to a second spinous process of the spinal column adjacent the first spinous process
the first implant being movable with the first spinous process independent of movement of the second implant.
